(19) United States
(12) Patent Application Publication
(10) Pub. No.: US 2015/0283400 A1
(43) Pub. Date: Oct. 8, 2015

(54) Control System for Therapeutic LED System in a Hot Tub or a Whirlpool Bathtub

(71) Applicant: David E. Hatley, Gray, TN (US)

(72) Inventor: David E. Hatley, Gray, TN (US)

(21) Appl. No.: 14/693,467

(22) Filed: Apr. 22, 2015

Related U.S. Application Data
(63) Continuation-in-part of application No. 14/266,312, filed on Apr. 30, 2014.
(60) Provisional application No. 61/982,539, filed on Apr. 22, 2014.
(60) Provisional application No. 61/817,641, filed on Apr. 30, 2013.

(51) Int. Cl. A61N 5/06; A61H 33/00

(52) U.S. Cl. CPC A61N 5/0618; A61H 33/00; A61N 2005/0626; A61N 2005/0642; A61N 2005/0652; A61N 2005/0668; A61N 2005/0663

(57) ABSTRACT

A system to electronically connect Light Emitting Diode perimeter lights installed around the upper inward-facing wall of a hot tub or whirlpool bathtub to an external control panel, allowing an occupant or user of the hot tub or whirlpool bathtub to dim or brighten the LED lights utilizing a button or dimmer switch on the external control panel. Said LED bulbs are capable of being adjusted to illuminate in different modes of different colors, different wavelengths, and different intensities, whereby light emitted by said LED bulbs in said different modes is adapted to aid in specific medical conditions and assist in maintaining healthy skin. A control system for adjusting the intensity of the light emitted by said LED bulbs includes a control panel with a user interface, positioned on a top surface of the tub.

### CROSS-REFERENCE TO RELATED APPLICATIONS

This Application claims the benefit of U.S. Provisional Patent Application Ser. No. 61/982,539, filed Apr. 22, 2014, the entirety of which is incorporated herein by reference. This Application is also a continuation-in-part of U.S. patent application Ser. No. 14/266,312, filed Apr. 30, 2014, which claims the benefit of U.S. Provisional Patent Application Ser. No. 61/817641, filed Apr. 30, 2013. All of the foregoing applications are incorporated herein by reference in their entirety.

### STATEMENT REGARDING FEDERALLY SPONSORED RESEARCH OR DEVELOPMENT

Not Applicable

### BACKGROUND OF THE INVENTION

1. Field of Invention

The present invention relates to control systems for hot tubs and whirlpools, and also to light therapy for treating symptoms of seasonal affective disorder.

2. Description of the Related Art

Hot tubs and similar devices are used for recreation, exercise, and physical therapy. Hot tubs are often employed for treating sore muscles or for relaxation after exercise or rigorous physical activity.

Use of hot tubs often occurs out of doors and after dark. For reasons of safety and convenience, lighting systems are often mounted in hot tubs. Such lighting systems help to create a positive atmosphere or ambience, which may change as a function of the brightness of the lights, the types and colors of the lights, the number of light sources, and the location of the light sources relative to the users.

Lighting systems in hot tubs or pools have historically involved placing point light sources slightly above the water line within the main body of the tub, generally in water tight fixtures that either slightly protrude from the walls of the hot tub or pool, or, more commonly, are recessed within a niche formed into the walls to which they are mounted. Light is typically supplied from an incandescent light bulb or a light emitting diode (LED) lamp placed in the fixture.

The lighting systems in the art generally involve one or more point light sources of varying brightness placed below the water line. These light sources propagate light into a body of water in a direction principally perpendicular to the wall on which the light source is mounted.

U.S. Pat. No. 7,712,913 discloses a lighted panel system for use in hot tubs, swim spas, and swimming pools that is mounted to the wall or lining of a tub. The lighted panel system comprises a light diffusing panel, a light source that illuminates the light diffusing panel, and a mounting structure that couples the light diffusing panel to the tub wall. Diffusers are preferably provided to the light diffusing panel in line with the light source and mounting structure to decoratively hide such components and further diffuse the light.

U.S. Pat. No. 7,204,602 discloses an LED light assembly, the light assembly comprising a reflective plate comprising a plurality of perforations formed therethrough the reflective plate, a plurality of LED bulbs wherein each LED bulb protrudes through a respective perforation of the plurality of perforations, and a control circuit selectively operable to produce a plurality of colored lights through the plurality of LED bulbs wherein the control circuit comprises a switch which when activated a defined number of times produces a plurality of at least light colors and light patterns wherein each of the plurality of at least light colors and light patterns are selected based on the defined number of times the switch is activated.

U.S. Pat. No. 6,752,517 discloses a lighting system suitable for chromatherapy includes a plurality of light fixtures mounted through walls of a tub basin to project different color light into the water in the tub. The light fixtures are operated by a central control unit and each includes a housing having a concave internal surface. An array of light emitting diodes that project different color light is mounted under a cap covering one end of the housing. A lens is threaded onto the opposite end of the housing from within the tub basin to secure the light fixture to the tub. The concave surface has the different color light projected on it, and reflects light out the light fixture in mixed fashion.

### SUMMARY OF THE INVENTION

The present general inventive concept encompasses, in some of its several example embodiments, a system that electronically connects Light Emitting Diode (LED) perimeter lights installed around the upper inward-facing wall of a hot tub or whirlpool bathtub to an external control panel, allowing an occupant or user of the hot tub or whirlpool bathtub to dim or brighten the LED lights utilizing a button or dimmer switch on the external control panel. This system is employed, for example, in a housing containing individual light therapy LED bulbs that can be adjusted to illuminate up to nine different modes of different colors and different levels of nanometers designed to aid in specific medical conditions and assist in maintaining healthy skin. It is designed to be installed in tanning beds and hot tub products at angles directly aimed at the facial region.

In some embodiments, a therapeutic light emitting diode lighting system for installation in a hot tub wall includes a light source comprising light emitting diode (LED) bulbs, said LED bulbs capable of being adjusted to illuminate in up to nine different modes of different colors and different wavelengths, whereby light emitted by said LED bulbs in said different modes is adapted to aid in specific medical conditions and assist in maintaining healthy skin, and a housing to hold said LED bulbs, said housing configured to angle said LED bulbs to direct light at the facial region of a person using the lamp.

In some embodiments, a therapeutic light emitting diode lighting system is installed in a raised molded area slightly above the main body of the hot tub, designed so that the lights are substantially at face level so that therapeutic lighting is directed at the facial areas of persons using the tub.

### BRIEF DESCRIPTION OF THE SEVERAL VIEWS OF THE DRAWING FIGURES

The above features, and other features and aspects of various example embodiments of the present general inventive concept, will become more apparent from examination of the drawing figures, in which:

FIG. 1 illustrates a hot tub with a series of Light Emitting Diode (LED) perimeter lights installed around the upper inward-facing wall of the tub according to one example embodiment of the present general inventive concept;

FIG. 2 is a close-up view of the hot tub shown in FIG. 1;

FIG. 3 is a second close-up view of the hot tub shown in FIGS. 1 and 2, showing a person using the hot tub and the LED bulbs directing therapeutic light at the person's facial area; and

FIG. 4 is a block diagram illustrating the relationship between several components within an example embodiment of the present general inventive concept.

### DETAILED DESCRIPTION OF THE INVENTION

The present general inventive concept encompasses, in some of its several example embodiments, a system that electronically connects Light Emitting Diode (LED) perimeter lights installed around the upper inward-facing wall of a hot tub or whirlpool bathtub to an external or topside control panel, allowing an occupant or user of the hot tub or whirlpool bathtub to dim or brighten the LED lights utilizing a button or dimmer switch on the topside control panel. This system (hereinafter a “dimmer switch control system”) is employed, for example, in a housing containing individual light therapy LED bulbs that can be adjusted to illuminate up to nine different modes of different colors and different levels of nanometers designed to aid in specific medical conditions and assist in maintaining healthy skin. It is designed to be installed in tanning beds and hot tub products at angles directly aimed at the facial region.

In some embodiments, a therapeutic light emitting diode lighting system for installation in a hot tub wall includes a light source comprising light emitting diode (LED) bulbs, said LED bulbs capable of being adjusted to illuminate in up to nine different modes of different colors and different wavelengths, whereby light emitted by said LED bulbs in said different modes is adapted to aid in specific medical conditions and assist in maintaining healthy skin, and a housing to hold said LED bulbs, said housing configured to angle said LED bulbs to direct light at the facial region of a person using the lamp.

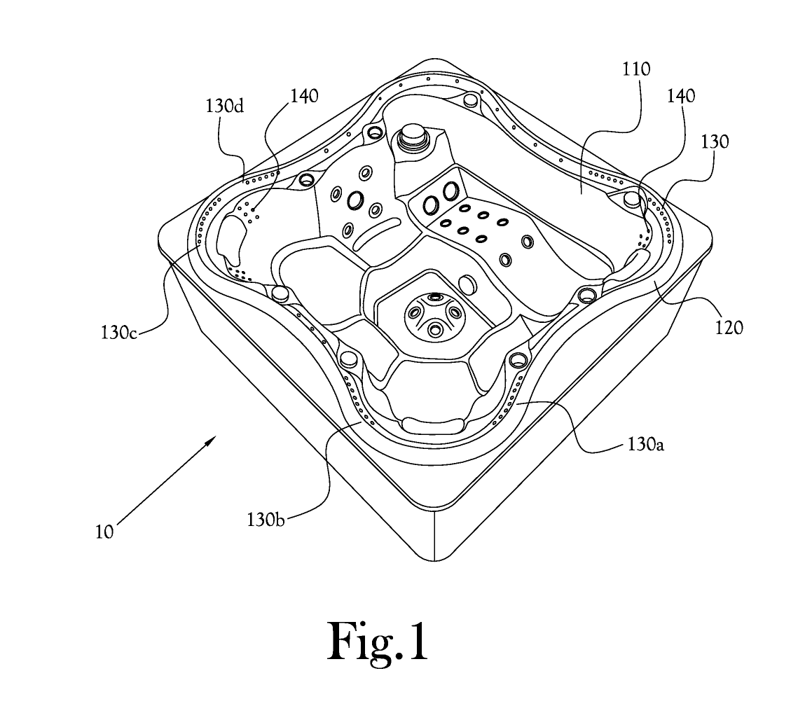

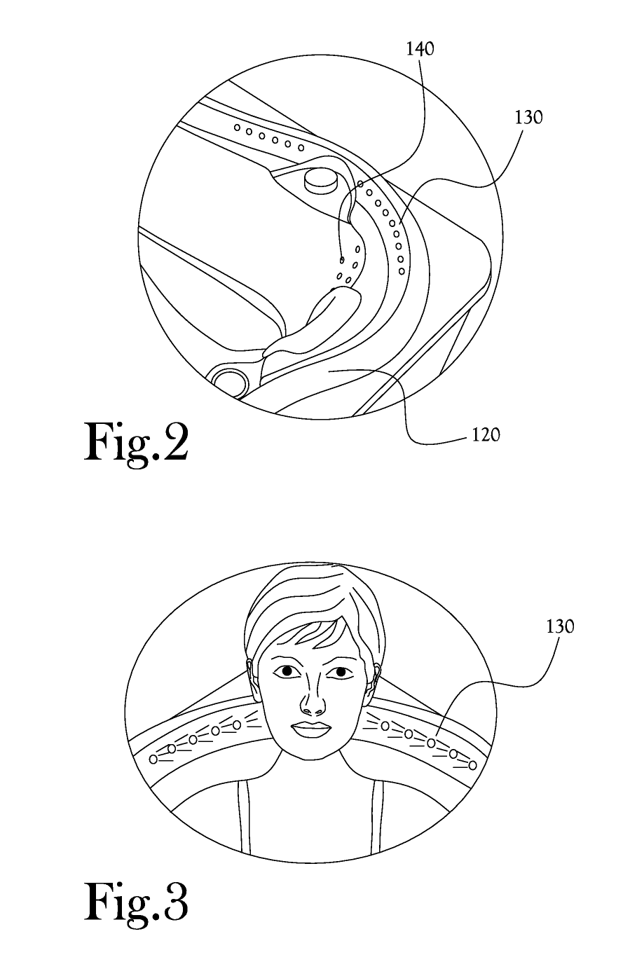

Turning to the Figures, FIG. 1 illustrates generally a hot tub comprising one example embodiment according to the present general inventive concept. As shown in FIG. 1, a hot tub 10 includes a molded tub body 110 with a lip or raised area 120. As shown in FIG. 1 and in the close-up view in FIG. 2, a grouping of LED bulbs 130 are positioned on the raised area 120. (In some embodiments, including the example embodiment shown in FIG. 1, the hot tub 10 is equipped with additional assemblies or groupings of LED bulbs are positioned in multiple locations on the raised area 120, e.g. LED bulb groupings 130a, 130b, 130c, 130d.) Generally, the raised area 120 and the grouping of LED bulbs 130 are a few inches above the water line of the hot tub 10 when the hot tub 10 is filled with water to the designed maximum capacity of the particular model of hot tub 10. In some embodiments, an additional grouping of LED bulbs 140 is positioned on the molded tub body 110, as shown in FIG. 2, below the above-mentioned grouping on LED bulbs 130 positioned on the raised area 120. (Generally, this second grouping of LED bulbs 140 is also above the water line of the hot tub 10.) As shown in FIG. 3, the grouping of LED bulbs 130 directs light generally at the facial area of a person sitting or reclining in the hot tub 10.

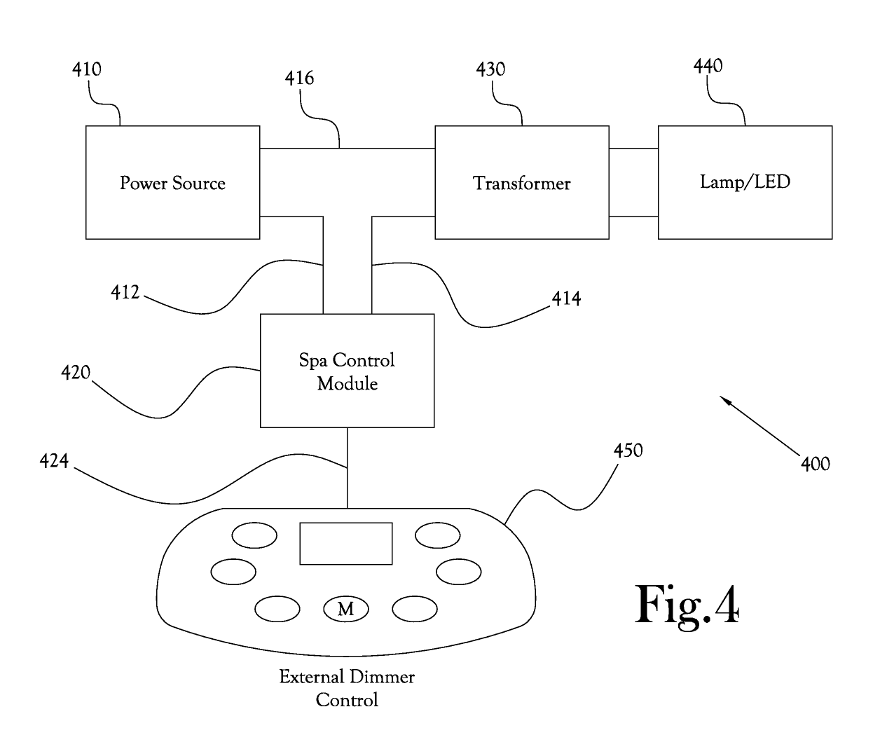

FIG. 4 is a block diagram illustrating generally a control system 400 for a therapeutic light emitting diode lighting assembly. As shown in FIG. 4, the system includes a power source 410, a spa control module 420, and a transformer 430 connected to a light source 440, generally an LED array or a lamp or some similar light element. The spa control module 420 is connected by power lines 412, 414 to the power source 410 and the transformer 430, respectively; the power source 410 and the transformer 430 also share a direct connection 416. The spa control module 420 is connected 424 to an external control panel 450, which includes means for adjusting the intensity and/or frequency of light emitted by the light source 440. The spa control module 420 accepts user commands entered into the external control panel 450, and spa control module 420 then adjusts the power and/or signals flowing to the transformer 430 and thence to light source 440.

In some embodiments, a therapeutic light emitting diode lighting system is installed in a raised molded area slightly above the main body of the hot tub, designed so that the lights are substantially at face level so that therapeutic lighting is directed at the facial areas of persons using the tub.

In some embodiments, a therapeutic LED bulb system is capable of nine different modes, including:
Mode 1: emitting Red light at 633 nanometers for aid in collagen and anti-aging.
Mode 2: emitting Blue light at 419 nanometers for aid in Acne treatment.
Mode 3: emitting Green light at 552 nanometers for aid in treatment for SAD or Seasonal Defect Disorder.
Mode 4: emitting normal Red light only intended for illumination.
Mode 5: emitting normal Blue light only intended for illumination.
Mode 6: emitting normal yellow light only intended for illumination.
Mode 7: emitting normal Green light only intended for illumination.
Mode 8: emitting White light only intended for illumination.
Mode 9: emitting normal Red, Blue, Yellow, Green, White light in a sequence of one color after the other intended only for illumination.

In some embodiments, a molded manifold for installation into the plumbing water line in a hot tub or whirlpool bathtub is fabricated from PVC or a similar polymer material.

Turning specifically to the dimmer switch control system, in some embodiments the system is wired and programmed into a main control pack that is used to operate every other function of the hot tub or whirlpool bathtub. Thus the occupant can use the same topside control to adjust the lighting level. In some embodiments, variable settings for the perimeter LED lighting controlled by the dimmer switch control system range from “off” to the highest intensity setting capable for the particular LED components in use.

In some embodiments of the present general inventive concept, a system for controlling the intensity of perimeter lighting in a hot tub or whirlpool bathtub includes a series of Light Emitting Diode (LED) perimeter lights installed around the upper inward-facing wall of a tub, a control module for adjusting the intensity of light emitted by said series of LED perimeter lights, said control module being in electronic communication with said LED perimeter lights, and an external control panel in communication with said control module, said external control panel being in electronic communication with said control module.

In some embodiments, the external control panel includes a button.

In some embodiments, the external control panel includes a dimmer switch.

In some embodiments of the present general inventive concept, a therapeutic light emitting diode lighting assembly with control system for use with tub includes a light source comprising light emitting diode (LED) bulbs, said LED bulbs capable of being adjusted to illuminate in different modes of different colors, different wavelengths, and different intensities, whereby light emitted by said LED bulbs in said different modes is adapted to aid in specific medical conditions and assist in maintaining healthy skin, a housing to hold said LED bulbs, said housing configured to angle said LED bulbs to direct light at the facial region of a person using the lamp, and a control system for adjusting the intensity of the light emitted by said LED bulbs, said control system including a control panel with a user interface, said control panel with a user interface being positioned on a top surface of the tub.

In some embodiments, said control system includes a button.

In some embodiments, said control system includes a dimmer switch.

The present general inventive concept thus provides a means for changing the setting of the lights themselves, for example, from a low-lighting to a higher-lighting setting. The present general inventive concept, in some of its several embodiments, allows for the creation of a custom environment for the occupant or user of the hot tub or whirlpool bathtub.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A system for controlling the intensity of perimeter lighting in a hot tub or whirlpool bathtub, comprising:
a series of Light Emitting Diode (LED) perimeter lights installed around the upper inward-facing wall of a tub;
a control module for adjusting the intensity of light emitted by said series of LED perimeter lights, said control module being in electronic communication with said LED perimeter lights; and
an external control panel in communication with said control module, said external control panel being in electronic communication with said control module.

2. The system of claim 1 wherein said external control panel includes a button.

3. The system of claim 1 wherein said external control panel includes a dimmer switch.

4. A therapeutic light emitting diode lighting assembly with control system for use with tub, comprising:
a light source comprising light emitting diode (LED) bulbs, said LED bulbs capable of being adjusted to illuminate in different modes of different colors, different wavelengths, and different intensities, whereby light emitted by said LED bulbs in said different modes is adapted to aid in specific medical conditions and assist in maintaining healthy skin;
a housing to hold said LED bulbs, said housing configured to angle said LED bulbs to direct light at the facial region of a person using the lamp; and
a control system for adjusting the intensity of the light emitted by said LED bulbs, said control system including a control panel with a user interface, said control panel with a user interface being positioned on a top surface of the tub.

5. The assembly of claim 4 wherein said control system includes a button.

6. The assembly of claim 4 wherein said control system includes a dimmer switch.
